(19) United States
(12) Patent Application Publication
(10) Pub. No.: US 2021/0098134 A1
(43) Pub. Date: Apr. 1, 2021

(54) MULTI-TASK LEARNING IN PHARMACOVIGILANCE

(71) Applicant: PricewaterhouseCoopers LLP, New York, NY (US)

(72) Inventors: Shinan ZHANG, New York, NY (US); Shantanu DEV, Chicago, IL (US); Joseph VOYLES, Louisville, KY (US); Anand S. RAO, Boston, MA (US); Matthew RICH, Chicago, IL (US); Siddhartha BHATTACHARYA, Philadelphia, PA (US)

(73) Assignee: PricewaterhouseCoopers LLP, New York, NY (US)

(21) Appl. No.: 17/032,608

(22) Filed: Sep. 25, 2020

Related U.S. Application Data
(60) Provisional application No. 62/907,349, filed on Sep. 27, 2019.
(60) Provisional application No. 62/907,229, filed on Sep. 27, 2019.

(51) Int. Cl. G16H 50/70; G16H 10/60; G16H 50/20; G16H 70/40; G16H 50/30; G16H 40/20; G06N 3/04; G06N 3/08; G06F 40/30; G06F 40/279

(52) U.S. Cl. CPC G16H 50/70; G16H 10/60; G16H 50/20; G16H 70/40; G16H 50/30; G16H 10/20; G06N 3/0445; G06N 3/08; G06F 40/30; G06F 40/279; G16H 40/20

(57) ABSTRACT

Techniques for pharmacovigilence adverse-event processing include receiving data comprising medical narrative text and generating, based on the received data, using a recurrent neural network encoder, a fixed-length context vector representation of the medical narrative text. The fixed-length context vector representation may then be queried, using a recurrent neural network decoder, to generate one or more hidden states. A first set of the one or more hidden states may be processed to generate an assessment of seriousness represented by the medical narrative text, and a second set of the one or more hidden states may be processed to generate a plurality of respective assessments of whether respective individual words of the medical narrative text correspond to one or more adverse events.

### CROSS-REFERENCE TO RELATED APPLICATIONS

This application claims the benefit of U.S. Provisional Application No. 62/907,229, filed Sep. 27, 2019, and of U.S. Provisional Application No. 62/907349, filed Sep. 27, 2019, the entire contents of each of which are incorporated herein by reference.

### FIELD

The present disclosure relates generally to machine learning in pharmacovigilence, and more specifically to multi-task learning (MTL) models in pharmacovigilence.

### BACKGROUND

Pharmacovigilance (PV) is the process of monitoring and assessing adverse drug reactions (ADRs) with pharmaceutical products. This also includes drug indication and medical errors since it is often linked with ADRs. The intent of PV is to assess ADRs so that investigations can be performed in determining how to mitigate or prevent risks for a given pharmaceutical product. Government regulatory agencies provide stringent guidelines for monitoring PV in order to ensure drug safety and quality.

The US Food and Drug Administration (FDA) has documented a significant increase in the case volume over recent years. In 2017, the FDA reported 1.8 million cases; ten years prior, total case volume was just 360,000. The FDA expects this number to continue to increase as additional pharmaceutical products are released into the market.

Natural Language Processing (NLP) in the medical literature space has been explored (see D. Demner-Fushman, W. Chapman, C. McDonald, “What can natural language processing do for clinical decision support?” J Biomed Inform. 2009; 425:76072.), but adoption of NLP systems, has been quite sparse across the healthcare sector, as evident in pharmacovigilance. A few clinical NLP systems have formalized information extraction from clinical texts (see E. Soysal, J. Wang, M. Jiang, Y. Wu, S. Pakhomov, H. Liu, H. Xu, “CLAMP—a toolkit for efficiently building customized clinical natural language processing pipelines”, JAMIA, Doi: 10.1093/jamia/ocx132; G. Savova, J. Masanz, P. Ogren, et al, “Mayo clinical Text Analysis and Knowledge Extraction System (cTAKES): architecture, component evaluation and applications”, J Am Med Inform Assoc. 2010; 175:50713). For example, relationship extraction, classification, and named entity recognition (NER) have been studied and have seen significant improvement over the years. For example, McCallum and Li were the first to use a conditional random field (CRF) based NER to annotate text. (See A. McCallum and W. Li, “Early Results for Named Entity Recognition with Conditional Random Fields,” Feature Induction and Web-Enhanced Lexicons. 2004, 10.3115/1119176.1119206.) With the advancement of deep learning, Collobert and Weston (2011) were some of the early adopters of using feed-forward and convolution neural networks for text annotation in order to achieve state of the art performance. (See R. Collobert and J. Weston, “A unified architecture for natural language processing: deep neural networks with multitask learning,” In Proceedings of the 25th international conference on Machine learning (ICML '08), 2008, ACM, New York, N.Y., USA, 160-167.) Huang, Xu, and Yu continued the research by utilizing recurrent neural networks (RNNs) with a CRF. (See Z. Huang, W. Xu, and K. Yu, “Bidirectional LSTM-CRF Models for Sequence Tagging,” 2015, arXiv preprint arXiv:1508.01991.) These works have served as a foundation for the exploration of applying NER in the medical domain. More recently, Jagannatha and Yu (2016) explored annotation of unstructured electronic health records (EHR) in order to identify entities such as ADRs, drug name, dosage, and disease. (See A. Jagannatha, H. Yu, “Structured prediction models for RNN based sequence labeling in clinical text,” Proc Conf Empir Methods Nat Lang Process. 2016; 2016:856-865.)

A neural network based approach to text classification is able to provide text compression that can be used as a context vector for transfer learning. (See M. Luong, Q. Le, I. Sutskever, O. Vinyals and L. Kaiser, “Multi-task Sequence to Sequence Learning,” Proceedings of ICLR, 2015.) This was made possible with the introduction of word embeddings as a way to encode text. Mikolov (2013) demonstrated that word embeddings are a method of representing the latent information surrounding a given word. (See Q. Le and T. Mikolov: “Distributed representations of sentences and documents,” In Proceedings of the 31st International Conference on Machine Learning (ICML-14), 2014, pages 1188-1196; T. Mikolov, I. Sutskever, K. Chen, G. S Corrado, and J. Dean: “Distributed representations of words and phrases and their compositionality,” In Advances in neural information processing systems, 2013, pages 3111-3119.) Zhang, Zhao, and LeCun demonstrated that convolutional neural networks (CNNs) and RNNs can be utilized to perform text classification. (See X. Zhang; J. Zhao; Y. LeCun, “Character-level Convolutional Networks for Text Classification,” Advances in Neural Information Processing Systems 28 NIPS, 2015.) In Pharmacovigilance, pre-trained word embeddings and recurrent neural networks have been shown to be effective in classifying case narratives. (See S. Dev, S. Zhang, J. Voyles and A. S. Rao, “Automated classification of adverse events in pharmacovigilance,” 2017 IEEE International Conference on Bioinformatics and Biomedicine (BIBM), Kansas City, Mo., 2017, pp. 1562-1566.)

Multi-task learning (MTL) is a subset within machine learning. A goal of multi-task learning is to provide a generalization within a subset of tasks through inductive transfer (transfer learning). This can be achieved by training several of the tasks in parallel. Caruna described MTL as shared representation, where information learned from a task can then be used to train another task. (See R. Caruana 1997. Multitask Learning. Mach. Learn. 28, 1 (July 1997), 41-75.) In addition, MTL also serves as an indirect form of regularization. (See O. Chapelle, P. Shivaswamy, S. Vadrevu, K. Weinberger, Y. Zhang, and B. Tseng, “Boosted multi-task learning,” 2011, Mach. Learn. 85, 1-2, 149-173.) With breakthroughs in transfer learning on computer vision, MTL is being further explored in the NLP space. For example, Collobert and Weston showed a unified NLP architecture which enabled MTL by sharing a lookup table (see R. Collobert and J. Weston, “A unified architecture for natural language processing: deep neural networks with multitask learning,” In Proceedings of the 25th international conference on Machine learning (ICML '08), 2008, ACM, New York, N.Y., USA, 160-167), and Hashimoto and Xiong were able to utilize MTL in NLP to perform tagging, parsing, relatedness, and entailment (see K. Hashimoto, C. Xiong, Y. Tsuruoka and R. Socher, “A Joint Many-Task Model: Growing a Neural Network for Multiple NLP Tasks,” 2017, EMNLP).

### SUMMARY

Pharmaceutical companies attempting to perform pharmacovigilence are struggling to keep up with the documented rise in case volume over recent years. According to known techniques, case ingestion and assessment are largely manual tasks, where clinicians and case managers have to individually examine a case. This approach is not scalable and continues to become more expensive.

One objective in Adverse Event (AE) triage is examining case reports for key AE descriptions and determining the seriousness of a case. Previous research has attempted to address the task of determining the seriousness of a case, which can be formalized as a text classification problem (see S. Dev, S. Zhang, J. Voyles and A. S. Rao, “Automated classification of adverse events in pharmacovigilance,” 2017 IEEE International Conference on Bioinformatics and Biomedicine (BIBM), Kansas City, Mo., 2017, pp. 1562-1566). However, determining case seriousness is only one of the many tasks that human case managers have to do during adverse events processing. Herein, techniques are set forth for performing multiple tasks required for AE processing using a unified multi-task learning system. As explained below, this disclosure sets forth machine learning algorithms designated to automate certain aspects of PV, including medical annotation, classification, and linking to medical terminology dictionaries.

Disclosed herein are systems, methods, and techniques for processing medical narrative text through a multi-task learning model to generate various outputs including at least (a) an assessment of seriousness of the case represented by the medical narrative text and (b) an assessment of whether individual words in the medical narrative text represent an adverse event. In some embodiments, the model may be further configured to generate, based on data regarding the assessment of whether individual words in the medical narrative text represent an adverse event, (c) a prediction of one or more dictionary terms (e.g., medical dictionary terms) most likely to be associated with an adverse event indicated by the medical narrative text.

In some embodiments, a pharmacovigilence adverse-event processing system is provided, the system comprising one or more processors configured to: receive data comprising medical narrative text; generate, based on the received data, using a recurrent neural network encoder, a fixed-length context vector representation of the medical narrative text; query the fixed-length context vector representation, using a recurrent neural network decoder, to generate one or more hidden states; process a first set of one or more of the one or more hidden states to generate an assessment of seriousness represented by the medical narrative text; and process a second set of one or more of the one or more hidden states to generate a plurality of respective assessments of whether respective individual words of the medical narrative text correspond to one or more adverse events.

In some embodiments of the system, generating the fixed-length context vector representation comprises using one or more distributed word representations.

In some embodiments of the system, generating the fixed-length context vector representation comprises: encoding the data comprising the medical narrative text using a word embedding to generate a vectorized representation of a plurality of individual words of the medical narrative text; and encoding a sequence of the word embedding, using a recurrent neural network encoder, into the fixed-length context vector representation.

In some embodiments of the system, the word embedding is pre-trained on a corpus of medical publication documents.

In some embodiments of the system, the assessment of seriousness comprises a binary indication of whether a case is a serious case or a non-serious case.

In some embodiments of the system, processing the first set of hidden states to generate the assessment of seriousness comprises: processing the first set of hidden states through a plurality of fully connected layers to generate a fully connected layers output; and processing the fully connected layers output using a first softmax function configured to generate a probability distribution indicating whether a case is serious or non-serious.

In some embodiments of the system, the plurality of fully connected layers comprise at least one dense layer followed by at least one dropout layer.

In some embodiments of the system, processing the first set of hidden states to generating the assessment of seriousness comprises applying a two layer feed-forward network with dropout.

In some embodiments of the system, processing the second set of hidden states to generate the plurality of respective assessments of whether respective individual words of the medical narrative text correspond to one or more adverse events comprises performing a sequence labeling task using the second set of hidden states as an input sequence for the sequence labeling task.

In some embodiments of the system, performing the sequence labeling task comprises determining, based on the input sequence, a label sequence having a highest probability.

In some embodiments of the system, performing the sequence labeling task comprises using a decoded hidden state at a plurality of steps and the fixed-length context vector representation to determine a label for one of the plurality of individual words.

In some embodiments of the system, processing the second set of hidden states to generate the plurality of respective assessments of whether respective individual words of the medical narrative text correspond to one or more adverse events comprises applying a second softmax function to the second set of hidden states of the recurrent neural network decoder.

In some embodiments of the system, processing the second set of hidden states to generate the plurality of respective assessments of whether respective individual words of the medical narrative text correspond to one or more adverse events comprises applying a bidirectional Long short-term memory (LSTM) network.

In some embodiments of the system, the one or more processors are configured to: based on one or more of the individual words determined to correspond to one or more adverse events, generate a prediction of set of dictionary terms.

In some embodiments of the system, generating the prediction of the set of dictionary terms comprises: generating character embeddings based on the one or more of the individual words determined to correspond to one or more adverse events; and process the character embeddings to generate the prediction of the set of dictionary terms.

In some embodiments of the system, processing the character embeddings to generate the set of dictionary terms comprises: processing the character embeddings through a set of parallel convolutional neural networks; applying respective sample-based discretization processes to outputs of the parallel convolutional neural networks; concatenating outputs of the discretization processes to generate concatenated data; processing the concatenated data through a series of fully-connected layers; and processing an output of the series of fully-connected layers through a third softmax function configured to generate the prediction of the set of dictionary terms.

In some embodiments of the system, the one or more processors are configured to compute a total loss based on a sum of a first loss and a second loss, wherein the first loss quantifies loss associated with the assessment of seriousness the second loss quantifies loss associated with the plurality of respective assessments of whether respective individual words of the medical narrative text correspond to one or more adverse events.

In some embodiments of the system, computing the total loss comprises computing the first loss using negative log-likelihood loss.

In some embodiments of the system, computing the total loss comprises computing the second loss using masked cross entropy loss.

In some embodiments of the system: the sum is further based on a third loss, wherein the third loss quantifies loss associated with the generation of the fixed-length context vector representation; and computing the total loss comprises computing the third loss using masked cross entropy loss.

In some embodiments, a pharmacovigilence adverse-event processing method is provided, the method performed at a system comprising one or more processors, the method comprising: receiving data comprising medical narrative text; generating, based on the received data, using a recurrent neural network encoder, a fixed-length context vector representation of the medical narrative text; querying the fixed-length context vector representation, using a recurrent neural network decoder, to generate one or more hidden states; processing a first set of one or more of the one or more hidden states to generate an assessment of seriousness represented by the medical narrative text; and processing a second set of one or more of the one or more hidden states to generate a plurality of respective assessments of whether respective individual words of the medical narrative text correspond to one or more adverse events.

In some embodiments, a non-transitory computer-readable storage medium storing instructions for pharmacovigilence adverse-event processing is provided, the instructions configured to be executed by one or more processors of a system to cause the system to: receive data comprising medical narrative text; generate, based on the received data, using a recurrent neural network encoder, a fixed-length context vector representation of the medical narrative text; query the fixed-length context vector representation, using a recurrent neural network decoder, to generate one or more hidden states; process a first set of one or more of the one or more hidden states to generate an assessment of seriousness represented by the medical narrative text; and process a second set of one or more of the one or more hidden states to generate a plurality of respective assessments of whether respective individual words of the medical narrative text correspond to one or more adverse events.

It will be appreciated that any of the aspects, features and options described in view of the system(s) apply equally to the method(s) and computer-readable storage medium(s), and vice versa. It will also be clear that any one or more of the above aspects, features and options can be combined. According to an aspect, any one or more of the characteristics of any one or more of the systems, methods, and/or computer-readable storage mediums recited above may be combined, in whole or in part, with one another and/or with any other features or characteristics described elsewhere herein.

### BRIEF DESCRIPTION OF THE FIGURES

Features will become apparent to those of ordinary skill in the art by describing in detail exemplary embodiments with reference to the attached drawings in which:

FIG. 1 depicts a system for multi-task learning in pharmacovigilance and adverse event processing, in accordance with some embodiments.

FIG. 2 depicts a flow chart representing a method for multi-task learning in pharmacovigilance and adverse event processing, in accordance with some embodiments.

FIG. 3 schematically depicts data processing for multi-task learning in pharmacovigilance and adverse event processing, in accordance with some embodiments.

FIG. 4 schematically depicts data processing for generating a prediction of one or more dictionary terms, in accordance with some embodiments.

FIG. 5 depicts a computer, in accordance with some embodiments.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

As explained in detail below, this disclosure sets forth systems, methods, and techniques for multi-task learning in pharmacovigilance and adverse event processing.

As explained below, a system may be configured to automatically process medical narrative text through a multi-task learning model to generate various outputs including at least (a) an assessment of seriousness of the case represented by the medical narrative text and (b) an assessment of whether individual words in the medical narrative text represent an adverse event. In some embodiments, the model may be further configured to generate, based on data regarding the assessment of whether individual words in the medical narrative text represent an adverse event, (c) a prediction of one or more dictionary terms (e.g., medical dictionary terms) most likely to be associated with an adverse event indicated by the medical narrative text.

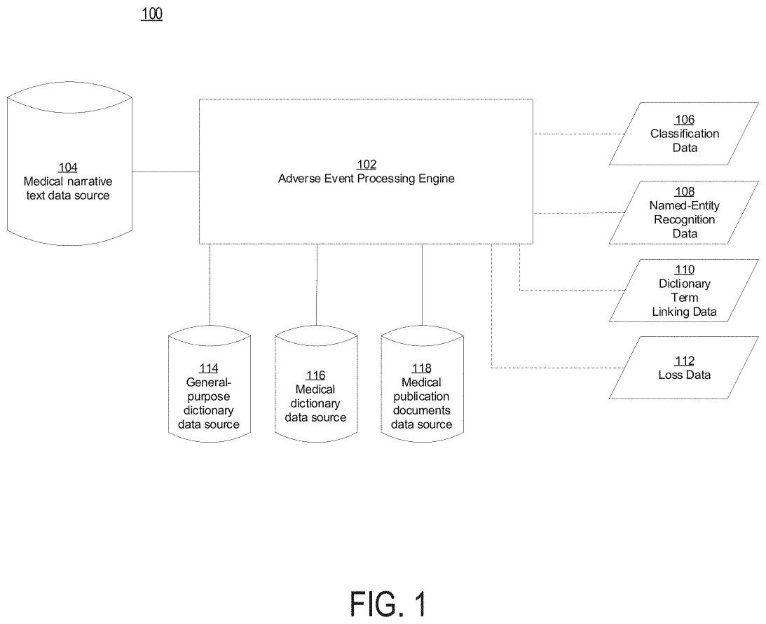

FIG. 1 depicts a system 100 multi-task learning in pharmacovigilance and adverse event processing, in accordance with some embodiments. As shown, system 100 may include adverse event processing engine 102, medical narrative text data source 104, general-purpose dictionary data source 114, medical dictionary data source 116, and medical publication documents data source 118. As further shown, adverse event processing engine 102 may be configured to generate one or more of classification data 106, named-entity recognition (NER) data 108, dictionary term linking data 110, and loss data 112. Each of the components of system 100 may be communicatively coupled with one or more of the other components such that they may send and receive electronic information via network communication amongst one another. For example, as shown by the lines in FIG. 1, engine 102 may be communicatively coupled with each of medical narrative text data source 104, general-purpose dictionary data source 114, medical dictionary data source 116, and medical publication documents data source 118.

In some embodiments, processing engine 102 may comprise one or more processors configured to receive input data, process said received data, and generate output data. For example, processing engine 102 may include one or more processors of a server system, distributed computing system, personal computer, laptop, and/or mobile electronic device.

In some embodiments, processing engine 102 may be configured to receive input receive input data from one or more of medical narrative text data source 104, general-purpose dictionary data source 114, medical dictionary data source 116, and medical publication documents data source 118. The input data received from one or more of data sources 104, 114, 116, and 118 may be processed by engine 102 to create, refine, update, and/or execute one or more algorithms or models to generate output data including but not limited to classification data 106, NER data 108, dictionary term linking data 110, and loss data 112.

In some embodiments, one or more of data sources 104, 114, 116, and 118 may comprise one or more electronic storage mediums configured to store and provide information to engine 102, whereby said provided information may be used as input data by engine 102 for execution of one or more of the pharmacovigilence and/or adverse event processing techniques described herein. One or more of data sources 104, 114, 116, and 118 may be configured to transmit data to engine 102 as part of batch uploads, uploads of individual data sets, as part of a user-directed upload, as part of an automated data scraping process, and/or as part of a periodic/scheduled upload process.

In some embodiments, medical narrative text data source 104 may comprise any one or more electronic storage mediums configured to store and provide data regarding medical narrative text. For example, data source 104 may comprise data (and/or associated metadata) comprising case narratives, which may be taken from [where do you get case narrative text to train the system? In actual implementation, where would the case narrative data be sourced from by end-users?]. In some embodiments, medical narrative text may be provided by data source 104 as a corpus of medical narrative texts (e.g., including thousands or millions of medical narrative texts). In some embodiments, medical narrative text may be provided by data source 104 as an individual medical narrative text, for example based on a medical narrative text manually inputted and/or uploaded by a user such as a medical practitioner.

In some embodiments, general-purpose dictionary data source 106 may comprise any comprise any one or more electronic storage mediums configured to store and provide data regarding dictionary information, such as words and the associated definitions. In some embodiments, general-purpose dictionary data may include definition information taken from one or more electronic (e.g., web-hosted) general-purpose dictionary resources.

In some embodiments, medical dictionary data source 108 may comprise any comprise any one or more electronic storage mediums configured to store and provide data regarding medical dictionary information, such as words and the associated definitions pertaining to medical science and/or reviewed and validated by medical professionals or academics. In some embodiments, medical dictionary data may include definition information taken from one or more electronic (e.g., web-hosted) medical dictionary resources. In some embodiments, medical dictionary data source 108 may comprise a clinically validated medical terminology dictionary and/or thesaurus. In some embodiments, medical dictionary data source 108 may comprise one or more dictionaries/thesauruses used by one or more regulatory authorities and/or pharmaceutical industry parties during the pharmaceutical regulatory process. In some embodiments, medical dictionary data source 108 may comprise one or more adverse event classification dictionaries. In some embodiments, medical dictionary data source 108 may comprise the Medical Dictionary for Regulatory Activities (MedDRA).

In some embodiments, medical publication documents data source 118 may comprise any one or more electronic storage mediums configured to store and provide data regarding medical publication documents. In some embodiments, the medical publication documents may include medical publication abstracts, medical publication full text, and/or medical textbook text. In some embodiments, medical publication documents may be sourced from one or more electronic (e.g., web-hosted) medical publication resources. In some embodiments, medical dictionary data source 108 may comprise a clinically validated medical terminology dictionary and/or thesaurus.

As stated above, engine 102 may process received data in accordance with one or more of the techniques described herein to generate output data including but not limited to classification data 106, NER data 108, dictionary term linking data 110, and/or loss data 112. Any or all output data generated by engine 102 may be stored locally and/or remotely, transmitted to another system component and/or to another system, displayed or otherwise output for user consumption, and/or further processed by one or more local and/or remote systems.

In some embodiments, classification data 106 may comprise data characterizing a seriousness level of a case based on input medical narrative text associated with the case. For example, classification data 106 may classify cases in a binary manner as either serious or not serious, for example by denoting a 1 for a serious case and a 0 for a non-serious case.

In some embodiments, NER data 108 may comprise data that classifies one or more words from the input medical narrative text into one or more predefined categories. In some embodiments, NER data 108 may comprise a word-by-word assessment for a plurality of words in the input medical narrative text indicating whether each of the individual words in the medical narrative text are respectively determined to be associated with one or more adverse events.

In some embodiments, dictionary term linking data 110 may comprise data that predicts a set of one or more dictionary terms determined to be associated with the one or more words of the medical narrative text that, as indicated by NEW data 108, are determined to be associated with one or more adverse events. In some embodiments, linking data 110 may comprise a set of general-purpose dictionary terms and/or standardized/medical dictionary terms that are predicted to be related to one or more terms indicated by NER data 108 as associated with an adverse event.

In some embodiments, loss data 112 may comprise data quantifying and/or characterizing a loss associated with assessment of seriousness of a case (e.g., associated with generating classification data 106), data quantifying and/or characterizing a loss associated with a named-entity recognition operation (e.g., associated with generating NER data 108), data quantifying and/or characterizing a loss associated with a medical dictionary linking operation (e.g., associated with generating dictionary term linking data 110), and/or data quantifying and/or characterizing a loss associated with generation of a fixed-length context vector representation (e.g., as discussed in further detail below).

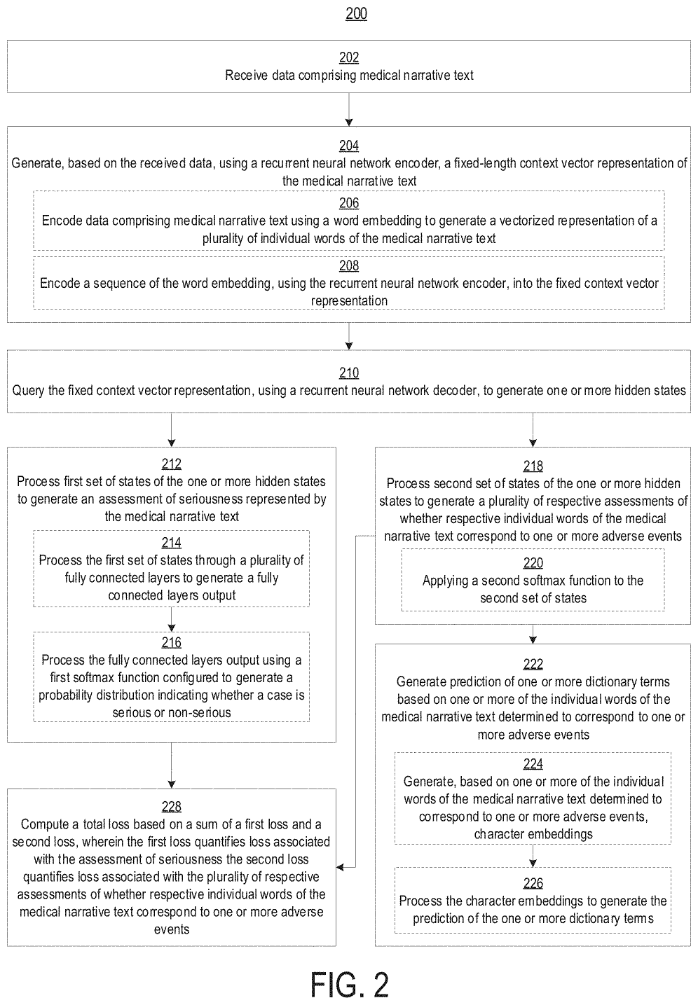

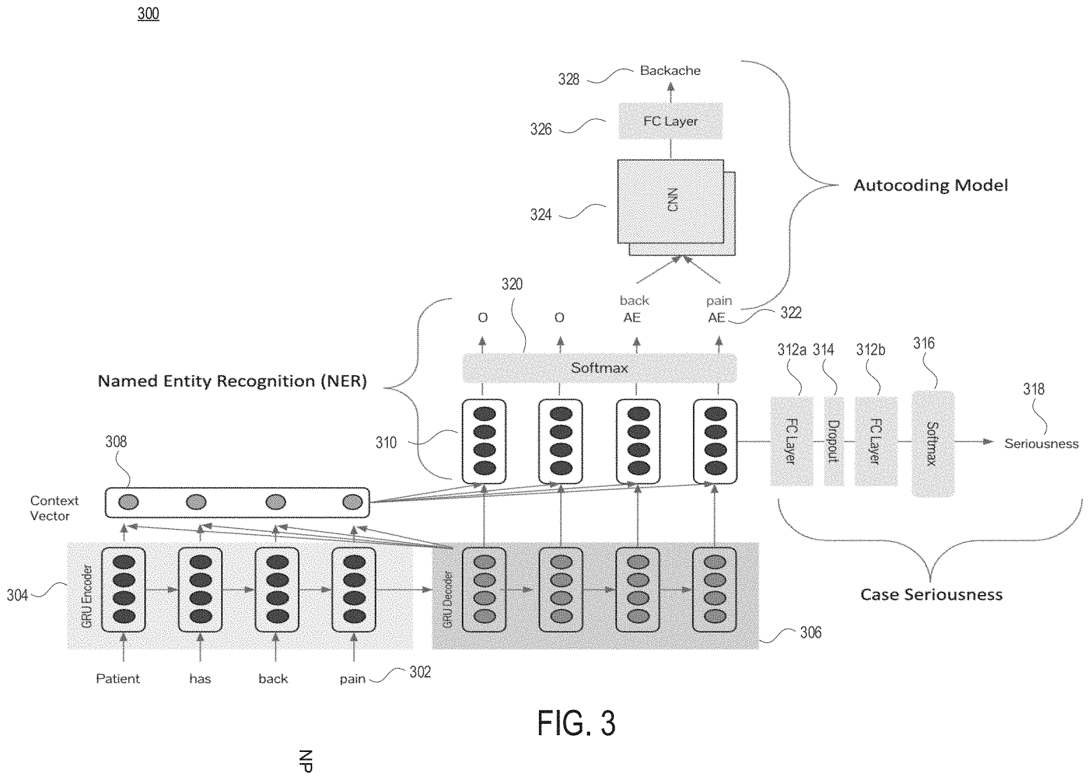

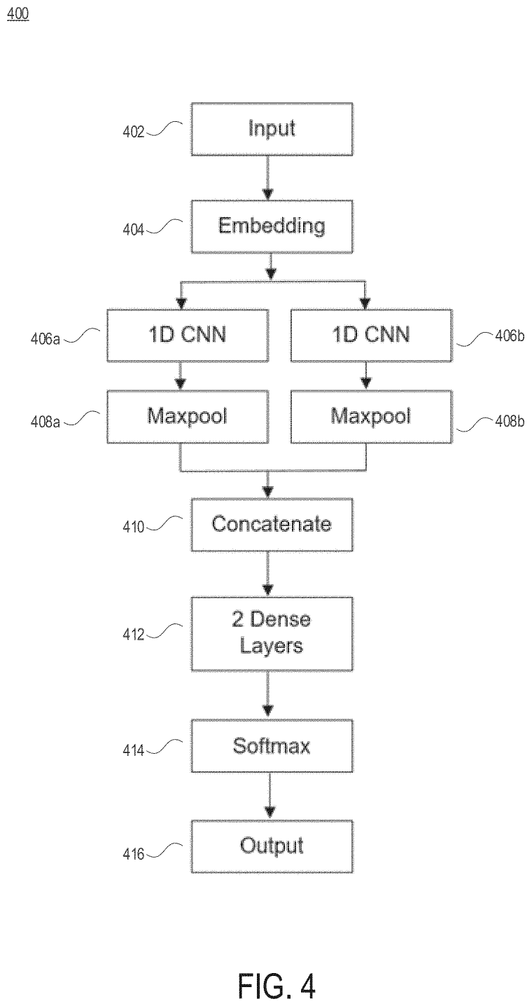

Techniques by which a system may use multi-task learning to process medical narrative text and to generate classification data, NER data, medical dictionary linking data, and/or loss data are described below with respect to FIGS. 2 and 3. FIG. 2 depicts a flow chart representing a method 200 for multi-task learning in pharmacovigilance and adverse event processing, in accordance with some embodiments. FIG. 3 schematically depicts data processing for multi-task learning in pharmacovigilance and adverse event processing, in accordance with some embodiments. FIG. 4 schematically depicts data processing for generating a prediction of one or more dictionary terms, in accordance with some embodiments. The data processing depicted in FIG. 3 and/or in FIG. 4 may correspond to data processing operations that are performed as method 200 is carried out. Throughout the description of method 200 below, reference to FIG. 3 and FIG. 4 is simultaneously made to aid understanding. In some embodiments, method 200 (and/or data processing as depicted in FIG. 3 and/or FIG. 4) may be performed by a system for multi-task learning in pharmacovigilance and adverse event processing, such as system 100.

At block 202, in some embodiments, the system may receive data comprising medical narrative text. In the example of system 100, adverse event processing engine 102 may receive (e.g., by wired or wireless network transmission) the data comprising the medical narrative text from medical narrative text data source 104.

In some embodiments, the system (e.g., system 100) may apply one or more data pre-processing and/or data preparation operations to the received data comprising the medical narrative text. For example, data preparation may comprise removal of one or more characters from the data comprising the medical narrative text. For example, removal of non-alphanumeric characters may be beneficial in some embodiments. ‘As Reported’ terms often contain special characters like ‘−’, ‘/’, ‘!’, etc., whose usage patterns may vary across case managers and data sets. Such variability in usage may lead to different vectored representations of similar ‘As Reported’ terms. Hence, dropping some or all non-alphanumeric characters from ‘As Reported Terms’ may help to eliminate these differences.

In some embodiments, data preparation may comprise removal of one or more words from the data comprising the medical narrative text. For example, removal of ‘stop words’ such as ‘a’, ‘an’, ‘at’, ‘and’, ‘be’, etc. may be beneficial. Words such as ‘a’, ‘an’, ‘at’, ‘and’, ‘be’, etc., are very common to ‘As Reported’ terms. These words may add very little value in distinguishing one ‘As Reported’ term from other. Hence, some or all of these words (and/or other similar words) may be removed from the vocabulary entirely.

In some embodiments, data preparation may comprise lemmatization of the data comprising the medical narrative text. Lemmatization is an algorithmic process of determining the lemma of a word based on its intended meaning. Lemmatization may be performed on input data to reduce morphological variations in the phrases to reduce the noise occurring because of usage of different forms of the same root word.

Below, blocks 204-220 describe how engine 102 may apply a multi-task learning model to process the received data comprising the medical narrative text to perform at least two tasks including generating, based on the medical narrative text, (a) an assessment of seriousness of a case represented by the text and (b) a plurality of respective assessments of whether respective individual words of the medical narrative text correspond to one or more adverse events. Blocks 222-226 describe performing a further operation based on the plurality of respective assessments of whether respective individual words of the medical narrative text correspond to one or more adverse events in order to predict a set of dictionary terms associated with an adverse event extracted from the input medical narrative text. Block 228 describes computation of one or more losses based on application of the multi-task learning model and performance of underlying tasks thereby.

Engine 102 may perform the operations described at blocks 204-224 (and 228) using a multi-task learning architecture such as data processing architecture 300 depicted schematically in FIG. 3. As shown in FIG. 3 and as described herein, a sequence-to-sequence (seq2seq) model with attention may be used to process medical narrative text and to generate the pharmacovigilence output data described herein. Sequence-to-sequence architectures have been successful in machine translation and other related NLP tasks (see M. Luong, H. Pham, C. Manning, “Effective Approaches to Attention-based Neural Machine Translation,” CoRR, 2015; M. Luong, Q. Le, I. Sutskever, O. Vinyals and L. Kaiser, “Multi-task Sequence to Sequence Learning,” Proceedings of ICLR, 2015). In some embodiments, an encoder-decoder framework is used to learn a conditional probability of mapping an input sequence x₁, . . . xₙ into an output sequence y₁, . . . yₘ. In some embodiments of the model architecture disclosed herein, the input sequence is mapped back to itself, and then the learned hidden layers and context vector are used to share between different NLP tasks, as shown in FIG. 3 and as described below with reference to FIGS. 2 and 3.

At block 204, in some embodiments, the system may generate, based on the received data comprising the medical narrative text, using a recurrent neural network encoder, a fixed-length context vector representation of the medical narrative text.

At block 206, in some embodiments, generating the fixed-length context vector representation may comprise encoding the data comprising the medical narrative text using a word embedding to generate a vectorized representation of a plurality of individual words of the medical narrative text. At block 208, in some embodiments, generating the fixed-length context vector representation may comprise encoding a sequence of the word embedding, using the recurrent neural network encoder, into the fixed-length context vector representation. In some embodiments, the system may encode individual words of the medical narrative text into respective word embeddings at block 206, and the system may then take a sequence of the word embeddings (e.g., based on a sentence in the medical narrative text formed by the words in the medical narrative text) and learn the fixed-length context vector based on the sequence of word-embeddings.

As shown in FIG. 3, system 100 may apply an encoder-decoder framework using encoder 304 and decoder 306 to process input narrative text data 302. As shown in FIG. 3, system 100 may process input narrative text to 302 to generate context vector 308.

In some embodiments, to vectorize the input medical narrative text data to generate the fixed-length context vector representation, a distributed word representation may be used. Learned word embeddings may preserve the semantic patterns in relation to each other. (See T. Mikolov, I. Sutskever, K. Chen, G. S Corrado, and J. Dean: “Distributed representations of words and phrases and their compositionality,” In Advances in neural information processing systems, 2013, pages 3111-3119.) In some embodiments of the present disclosure, an embedding may be defined as a matrix Wₑ ∈ [symbol] ^{V×d}, where V ∈ [symbol] is the size of the vocabulary and d ∈ [symbol] is the dimension of the embedding. In some embodiments, one or more word embeddings pre-trained on medical publication documents (e.g., Medline/PubMed abstracts), such as documents sourced from medical publication documents data source, may be used.

In some embodiments, a Gated Recurring Unit (GRU) Encoder part of the network (e.g., GRU encoder 304) may follow the set of equations used in D. Bandanau, K. Cho, and Y. Bengio. “Neural machine translation by jointly learning to align and translate,” 2014, arXiv preprint arXiv:1409.0473 and Z. Yang, D. Yang, C. Dyer, X. He, A. Smola and E. H. Hovy, “Hierarchical Attention Networks for Document Classification,” 2016, Proceedings of the HLT-NAACL conference. San Diego, Calif., as shown below:

rₜ=σ(Wᵣxₜ+Uᵣhₜ₋₁+bᵣ) (1)

zₜ=σ(W_{z}xₜ+U_{z}hₜ₋₁+b_{z}) (2)

\tilde{h}ₜ=tanh(Wₕxₜ+rₜ⊙(Uₕhₜ₋₁)+bᵣ) (3)

hₜ=(1−zₜ)⊙h₍ₜ₋₁₎+zₜ⊙\tilde{h}ₜ (4)

where hₜ is the hidden state at time t, xt is the input at time t, h₍ₜ₋₁₎ is the hidden state of the previous layer at time t−1 or the initial hidden state at time 0, and rₜ, zₜ, \tilde{h}ₜ are the reset, update, and the candidate hidden state, respectively. σ is the sigmoid function.

At block 210, in some embodiments, the system may query the fixed-length context vector representation, using a recurrent neural network decoder, to generate a plurality of hidden states. The recurrent neural network decoder may, for example, be GRU decoder 306 in FIG. 3. Following block 210, the data processing flow may bifurcate such that the system may perform one task (e.g., a classification task) on the basis of processing the output of the decoder in a first manner (e.g., by processing a first set of one or more of the one or more hidden states) and may perform a second task (e.g., an NER task) on the basis of processing the output of the decoder in a second manner (e.g., by processing a second set of one or more of the one or more hidden states). Below, blocks 212-216 describe performing a seriousness classification task on the basis of the output of the decoder, and blocks 218-224 describe performing an NER classification task on the basis of the output of the decoder.

At block 212, in some embodiments, the system may process one or more of the one or more hidden states to generate an assessment of seriousness represented by the medical narrative text. In some embodiments, processing one or more of the one or more hidden states may comprise processing a first set of states of the one or more hidden states. In some embodiments, processing the first set of states may comprise processing a single hidden state from the one or more hidden states generated by the decoder. In some embodiments, processing the first set of states may comprise processing a final (e.g., last) hidden state from the one or more hidden states generated by the decoder. In some embodiments, processing the first set of states may comprise applying one or more dropouts, one or more fully connected layers, and/or one or more softmax functions to generate the assessment of seriousness.

At block 214, in some embodiments, processing the first set of states to generate the assessment of seriousness may comprise processing the first set of states through a plurality of fully connected layers to generate a fully connected layers output.

At block 216, in some embodiments, processing the first set of states to generate the assessment of seriousness may comprise processing the fully connected layers output using a first softmax function configured to generate a probability distribution indicating whether a case is serious or non-serious.

As shown in FIG. 3, system 100 may process data 310 (e.g., data including one or more hidden states) through fully-connected layers 312a and 312b and through dropout layer 314 between the two fully-connected layers. System 100 may then process the output from fully-connected layer 321b through softmax function 316 to generate seriousness assessment data 318.

Identifying whether a case narrative is serious or non-serious may be an important aspect of in PV, in some embodiments. In fact, the FDA provides a broad outline of the definition of what is a serious case. (See U.S. Food and Drug Administration. “What is a Serious Adverse Event?”, U.S. Food and Drug Administration Home Page, 2016.) According to known techniques, case managers and medical reviewers have to manually read each individual case and flag the serious ones. One functionality of the systems and techniques using the models disclosed herein is to automate and flag serious cases with minimal intervention. For example, for the labels, a 1 may be denoted for a serious case and a 0 for a non-serious case.

In a similar manner to performing the NER classification task described with reference to blocks 218-220 below, after receiving the concatenated output features from the decoder, the system may process the concatenated output features through one or more layers. In some embodiments, the concatenated output features may comprise information from the hidden states of the decoder and the attention decoder states combined together. In some embodiments, the system may process the concatenated output features by applying two dense layers with a dropout applied in between the two dense layers for regularization. The last dense layer may, in some embodiments, compress the features to the two classes (e.g., serious/non-serious) where a log softmax may be applied. In some embodiments, the softmax layer may normalize the output of the fully connected layers into a probability distribution of the predicted number of classes (e.g., a first class for serious cases and a second class for non-serious cases).

In some embodiments, for a classification task such as the one described with reference to blocs 212-216, a two layer feed-forward network with dropout may be used as a baseline model.

At block 218, in some embodiments, the system may process one or more of the one or more hidden states to generate a plurality of respective assessments of whether respective individual words of the medical narrative text correspond to one or more adverse events. In some embodiments, processing one or more of the one or more hidden states may comprise processing a second set of states of the one or more hidden states. In some embodiments, the second set of states may have one or more overlapping states with the first set of states. In some embodiments, processing the second set of states may comprise processing a plurality of states using one or more softmax functions with respect to a plurality of time steps (e.g., with respect to every time step).

At block 220, in some embodiments, processing the second set of states to generate the plurality of respective assessments of whether respective individual words of the medical narrative text correspond to one or more adverse events may comprise applying a second softmax function to the second set of states.

In some embodiments, identifying adverse events in medical narrative text may be performed as a sequence labeling task that can be viewed as Named Entity Recognition (NER) as described herein. Give input sequence s, one may try to find the label sequence l with the highest probability.

In some embodiments, the decoded hidden state at each step and the context vector may be used to classify to a label for the current word.

lₜ=softmax(tanh W_{c}[hₜ:cₜ])

where the context vector cₜ is calculated by the following attention mechanism (see M. Luong, H. Pham, C. Manning, “Effective Approaches to Attention-based Neural Machine Translation,” CoRR, 2015):

$$\begin{matrix}{∝}_{t} = f ( {h}_{t} , {\overline{h}}_{s} ) & ( 5 ) \\ {\overline{∝}}_{ts} = softmax ( {∝}_{t} ) & ( 6 ) \\ {c}_{t} = {\sum }_{t = 0}^{n} {\overline{∝}}_{ts} {\overline{h}}_{s} & ( 7 )\end{matrix}$$

where the scoring function ƒ compares the current target hidden state hₜ with each source hidden state h̅ₛ. Here, the general scoring function ƒ(hₜ,h̅ₛ)=hₜ^{T}Wₐh̅ₛ may be used (see id.) and ∝̅ₜₛ may be the attention weights.

As shown in FIG. 3, system 100 may process data 310 (e.g., data including one or more hidden states) through softmax function 320 to generate adverse event assessment data 322. While softmax function 316 may be configured to generate one assessment based on all of the words of the medical narrative text represented by data 310, softmax function 320 may be configured to generate a separate assessment for each work of the medical narrative text represented by data 310. As shown in FIG. 3, adverse event assessment data 322, generated by softmax function 320, indicates that the words “patient” and “has” do not correspond to adverse events whereas the words “back” and “pain” do correspond to adverse events.

In some embodiments, for an NER task such as the one described with reference to blocks 218-220, a bidirectional Long short-term memory (LSTM) network may be used as a baseline model.

In some embodiments, output data from an NER task such as the one described with reference to blocks 218-220 may be used to generate a prediction of one or more dictionary terms based on one or more of the individual words of the medical narrative text determined to correspond to one or more adverse events, for example as described below including with reference to blocks 222-226 of FIG. 2, elements 324-328 of FIG. 3, and FIG. 4. In some embodiments, generating a prediction of one or more dictionary terms based on one or more of the individual words of the medical narrative text determined to correspond to one or more adverse events may be performed as an auto coding task.

At block 222, in some embodiments, the system may generate a prediction of one or more dictionary terms based on one or more of the individual words of the medical narrative text determined to correspond to one or more adverse events.

At block 224, in some embodiments, generating the prediction of one or more dictionary terms based on the one or more of the individual words of the medical narrative text determined to correspond to one or more adverse events may comprise generating, based on one or more of the individual words of the medical narrative text determined to correspond to one or more adverse events, character embeddings.

At block 226, in some embodiments, generating the prediction of one or more dictionary terms based on the one or more of the individual words of the medical narrative text determined to correspond to one or more adverse events may comprise processing the character embeddings to generate the prediction of the one or more dictionary terms.

In some embodiments, once one or more words associated with an adverse event have been identified as explained above at blocks 218-220, the system (e.g., system 100) may perform an additional (and in some embodiments final) task to use the extraction identifying the adverse event and to encode the text into a character embeddings that may be processed to generate a predicted set of one or more dictionary terms, such as one or more standardized and/or medical dictionary terms (e.g., MedDRA terms) predicted to be associated with the adverse event indicated by the medical narrative text.

One embodiment of data processing for generating a prediction of one or more dictionary terms based on one or more of the individual words of the medical narrative text determined to correspond to one or more adverse events is shown by FIG. 3. As shown in FIG. 3, in some embodiments, adverse event assessment data 322 may be processed through an autocoding model including by processing said adverse event assessment data 322 through a set of parallel convolutional neural networks 324. In some embodiments, the CNNs in the set of parallel CNNS 324 may be CNNs of different filters. The output from the convolutional neural networks may then be processed through fully-connected layer 326 in order to generate output data 328, wherein output data 328 may comprise the prediction of the set of one or more dictionary terms (e.g., “backache,” as shown in FIG. 3). Output data 328 may share any one or more characteristics in common with linking data 110 described above with respect to FIG. 1 and/or with output data 416 described below with respect to FIG. 4.

One embodiment of data processing for generating a prediction of one or more dictionary terms based on one or more of the individual words of the medical narrative text determined to correspond to one or more adverse events is shown by FIG. 4. As shown in FIG. 4, in some embodiments, generating the set of dictionary terms may begin with input data 402 being processed to generate character embeddings at embeddings layer 404. Input data 402 may be data comprise output data from an NER as described herein, e.g., data indicating a plurality of respective assessments of whether respective individual words of a medical narrative text correspond to one or more adverse events. In some embodiments, the character embeddings layer 404 in the architecture depicted in FIG. 4 may internally learn the vectorized representations for characters.

The generated character embeddings from embeddings layer 404 may then be processed through a set of two or more parallel convolutional neural networks 406a and 406b. In some embodiments, convolutional neural networks 406a and 406b may be CNNs of different filters. In some embodiments, one both of CNN's 406a and 406b may be a one-dimensional (1D) convolution, which may capture sub-word information from the input text. In some embodiments, CNN's 406a and 406b may have different kernel sizes.

The system may then apply respective sample-based discretization processes (e.g., maxpooling) 408a and 408b in parallel to the output from each of the convolutional neural networks 406a and 406b. In some embodiments, a sample-based discretization process such as maxpooling may down-sample an input representation (e.g., image, hidden-layer output matrix, etc.), reducing its dimensionality and allowing for assumptions to be made about features contained in the sub-regions binned. A sample-based discretization process such as maxpooling may highly activate one or more key sub-words suitable for making the prediction of associated dictionary terms.

The system may then concatenate the outputs from the sample-based discretization processes 408a and 408b (e.g., concatenate the maxpooled data) as shown at concatenation operation 410.

The system may then process the concatenated data through a series of fully-connected layers (e.g., dense layers) 412. The series of fully-connected layers (e.g., dense layers) may generate complex features by performing non-linear combinations over the (concatenated) discretization (e.g., maxpool) output.

The system may finally process an output of the series of fully-connected layers 412 through a softmax function 414 configured to generate output data 416 comprising the prediction of the set of dictionary terms. In some embodiments, the softmax function 414 may be configured to generate a prediction of a number of dictionary terms that are determined to be most closely related; in some embodiments, the function 414 may be configured to predict a predetermined (or user specified, or dynamically determined) number of dictionary terms, such as the three closest terms or the five closest terms. In some embodiments, the function 414 may be configured to generates a probability distribution over the LLTs, such that the LLTs corresponding to the top scores (e.g., top three scores, top five scores, etc.) may then be recommended to a user.

Output data 416 may share any one or more characteristics in common with linking data 110 described above with respect to FIG. 1 and/or with output data 328 described above with respect to FIG. 3.

In some embodiments, generating a prediction of one or more dictionary terms based on one or more of the individual words of the medical narrative text determined to correspond to one or more adverse events may be performed as an auto coding task. In some embodiments, an auto coding model may allow users to verify and update a determination as needed for encoding adverse events to dictionary data, such as for encoding adverse events to the MedDRA hierarchy. In some embodiments, the auto coding model may be a machine learning model with human oversight, in which some or all ML model predictions may be reviewed and updated by a user before a case is ready for submission.

In some embodiments, a process of encoding adverse events to a dictionary (e.g., to the MedDRA dictionary) may be performed as a multi-class classification problem with ‘As Reported’ terms as the input and ‘Lower Level Terms (LLTs)’ as the target. In some embodiments, high variation in the target variable may lead to very few training examples per class, making it difficult for machine learning models to learn from past data. To address this problem, the ‘As Reported’, ‘To Be Coded’ and ‘Lower Level Terms’ may be combined as the input variable and the corresponding LLTs as the target variable. This may provide more training examples and help capture more technical terms from the LLTs, which otherwise couldn't be captured with just ‘As Reported’ terms.

For feature engineering for the auto coding model used to encode adverse events to a dictionary, text-based data from a labeled dataset may be converted into vectors in order to make the dataset machine readable. This conversion may enable selected features from the data to facilitate the machine learning process. Various text vectorization approaches may be used. In some embodiments, a Term Frequency-Inverse Document Frequency (TF-IDF) vectorization approach may be used with a K-Nearest Neighbor (KNN) model.

TF-IDF may use text vectorization techniques to identify the importance of a word in a corpus. This importance may be determined by the number of times the word appears in a document and may be offset by the frequency of the documents containing the word in the corpus. TF-IDF may automatically readjust feature weights based on the number of times the word appeared in the corpus.

A KNN model may be a non-parametric model used for classification and regression. A KNN model may search for k closest examples in a training set, which in turn may be used for making a prediction. In case of a classification problem, a KNN model may look at the frequency of each of the target labels in these k examples and may return the class with highest frequency as the output. In some embodiments, a KNN model may not be capable of sharing feature representation (e.g., sharing feature representation of medical narrative text) in the way that a deep learning model (e.g., as shown and described with reference to FIG. 4) is able to do.

In some embodiment, a deep learning model may be a more sophisticated approach to text vectorization that learns the features itself from the sequence of words (text input). In some embodiments, deep learning may works off the premise that rather than spending time selecting significant features, a computer may use a very large dataset to instead identify patterns that exist in the dataset and apply a series of transformation to those input features until finally the computer outputs a single end classification score. In some embodiments, a deep learning model such as the model shown and described with reference to FIG. 4 may be capable of sharing a feature representation (e.g., sharing feature representation of medical narrative text) and/or sharing hidden states.

In some embodiments, training of an auto coding model (e.g., a deep learning model) to be used to encode adverse events to a dictionary may comprise splitting a corpus of data into a training data set to be used to train the model and a test data set that will be unseen by the model during training and then used to test the model's performance. In some embodiments, any suitable split of the data corpus may be used; in some embodiments, a majority of the data may be used for training and a minority of the data may be used for testing. In some embodiments, a 90%-10% split may be used whereby 90% of the corpus is used for training and the remaining 10% is used for testing. The model may then be trained using the training data and may then be evaluated using the testing data to generate performance metrics.

In some embodiments, there may be class imbalance between the training and test sets. In order to reduce the impact of such a class imbalance on the model's performance, the distribution of sub-classes within the training and test sets may need to remain the same or similar. Hence, stratified sampling based on the Event Body System may be performed.

For a deep learning model, the data may be split into a training data set and a test/validation data set. In each pass of the training data, the model may be updated, and the model's performance may be tested on the test/validation data. This process may be repeated several times until no further improvement (or minimal further improvement, e.g., improvement below a predefined or dynamically determined performance metric threshold) is obtained in the performance on the test/validation data.

In some embodiments, a training data set for an auto coding model (e.g., a deep learning model) to be used to encode adverse events to a dictionary may comprise a sequence of tokens (words) from ‘As Reported’ terms as input (in some embodiments, after removal of non-alphanumeric characters as described above and/or converting all characters to lowercase) and the corresponding Lower Level Terms (LLTs) as target. One model architecture is shown by the data processing architecture depicted in FIG. 4 and described above with reference to FIG. 4.

In some embodiments, in order to enable efficient memory utilization and to accelerate the process of training the model, the model may be trained in batches, for example in batches of size 512 on GPU. Due to the very large number of trainable parameters in the network, it is often possible to overfit the model, and hence regularization techniques may be used. In some embodiments, techniques for regularization may include L2 regularization and dropout. Regularization is a technique to discourage the complexity of the model, and it may achieve this by penalizing a loss function. This may help to solve overfitting problems. Dropout refers to ignoring units (e.g., neurons) during the training phase of certain sets of units which may be chosen at random.

Returning to FIG. 2, the system (e.g., system 100) may in some embodiments be configured to compute a loss function characterizing loss of one or more models applied by the system. At block 228, in some embodiments, the system may compute a total loss based on a sum of a first loss and a second loss, wherein the first loss quantifies loss associated with the assessment of seriousness the second loss quantifies loss associated with the plurality of respective assessments of whether respective individual words of the medical narrative text correspond to one or more adverse events.

In some embodiments, for an autoencoder part (e.g., blocks 204-210) and/or for an NER part (e.g., blocks 218-220) of the network, a masked cross entropy loss may be used. Use of a masked cross entropy loss for these parts of the network may be appropriate because mini-batches during training may have been padded.

In some embodiments, for the classification task (e.g., blocks 212-216), negative log-likelihood loss may be used.

In some embodiments, a multi-task learning network such as those described herein may be trained end-to-end with three tasks (autoencoder, NER, and classification). In some embodiments, a multi-task learning network such as those described herein may be trained end-to-end with four tasks (autoencoder, NER, classification, and autocoding (e.g., linking to a dictionary such as MedDRA)). The overall loss is the summation of some or all of the tasks' losses.

In some embodiments, a multi-task learning model such as those disclosed herein may be expanded upon by adding one or more additional tasks. In some embodiments, an MTL model may extract Adverse Events (AE) from a case narrative and classify the entire narrative as serious or non-serious. However, case managers, in PV, in some embodiments, have to go a step further. After the case manager identifies an AE from a narrative, it also may have to classify seriousness at an AE level. This is a challenging problem to tackle from a machine learning standpoint because it may require that a determination be made from the extraction of a few words (e.g., just 2-3 words). Actual case managers may utilize contextual meaning of the entire case narrative to evaluate the seriousness at an AE level. Multi-task learning is a unique modeling approach for simulating how a case manager would handle AE level classification. Through the learned context vector (e.g., see FIG. 3), an additional classification task may be added that is able to inject a portion of the context and incorporate it with the NER AE labeled outputs in order to determine seriousness. Finally, in some embodiments, pre-trained word embeddings, such as the Medline vectors (see, e.g., S. Dev, S. Zhang, J. Voyles and A. S. Rao, “Automated classification of adverse events in pharmacovigilance,” 2017 IEEE International Conference on Bioinformatics and Biomedicine (BIBM), Kansas City, Mo., 2017, pp. 1562-1566) may be incorporated.

Disclosed herein, for example as explained above (and as further demonstrated by the Examples below) are novel models in PV for handling different tasks (e.g., annotation, classification) end-to-end. In some embodiments, a single network architecture may be utilized, where shared hidden weights may be utilized to process the same text in a set of different tasks. This disclosure demonstrates both the feasibility and practicality of using this modeling approach. In some embodiments, MTL is thus a useful regularization approach as evident from the disclosures herein (see, e.g., O. Chapelle, P. Shivaswamy, S. Vadrevu, K. Weinberger, Y. Zhang, and B. Tseng, “Boosted multi-task learning,” 2011, Mach. Learn. 85, 1-2, 149-173).

The models disclosed herein may also utilize, in some embodiments, an attention mechanism. In some embodiments, this may help provide explainability and transparency in the model. In PV, explainability may be important because case managers and regulators may need to have trust in deep learning models.

A failure to properly report AEs may lead to punitive fines. DARPA has nearly doubled their investment in explainable AI (XAI) research between 2017 and 2018. (See Defense Advanced Research Projects Agency (DARPA) Justification Budget Estimates for FY2019. February 2018. Page 116.) Hence, it may be expected that XAI may continue being a valuable component in the future of MTL research.

The disclosure herein shows that MTL may be an effective approach for solving PV tasks. As case manager's roles continue to evolve, the modular framework of MTL may thus be a useful method of handling the increase in case volume.

### Exemplary Computer

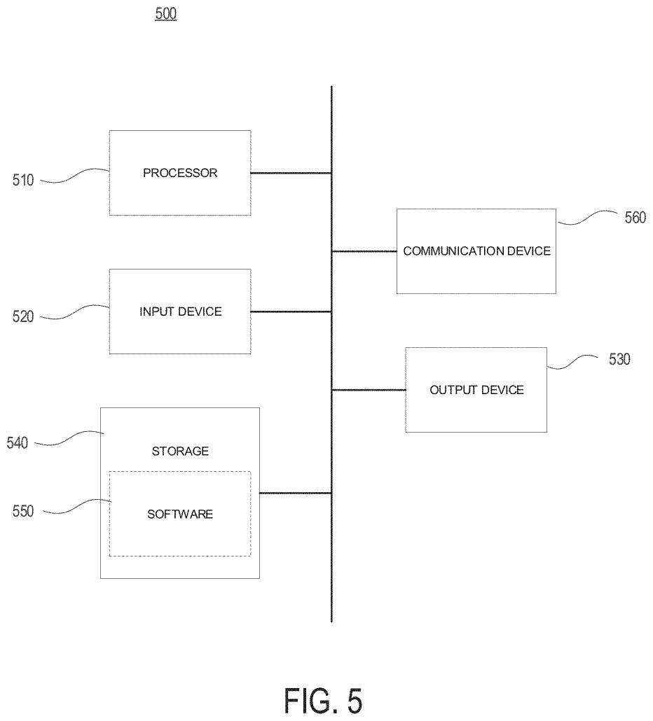

FIG. 5 illustrates a computer, in accordance with some embodiments. Computer 500 can be a component of a system for multi-task learning in pharmacovigilance and adverse event processing, such as system 100 and/or any of its subcomponents described above with respect to FIG. 1. In some embodiments, computer 500 may be configured to execute a method for multi-task learning in pharmacovigilance and adverse event processing, such as all or part of method 200 as described above with respect to FIG. 2. In some embodiments, computer 500 may be configured to execute data processing, in whole or in part, as depicted by the model architectures shown in one or both of FIGS. 3 and 4.

Computer 500 can be a host computer connected to a network. Computer 500 can be a client computer or a server. As shown in FIG. 5, computer 500 can be any suitable type of microprocessor-based device, such as a personal computer; workstation; server; or handheld computing device, such as a phone or tablet. The computer can include, for example, one or more of processor 510, input device 520, output device 530, storage 540, and communication device 560.

Input device 520 can be any suitable device that provides input, such as a touch screen or monitor, keyboard, mouse, or voice-recognition device. Output device 530 can be any suitable device that provides output, such as a touch screen, monitor, printer, disk drive, or speaker.

Storage 540 can be any suitable device that provides storage, such as an electrical, magnetic, or optical memory, including a RAM, cache, hard drive, CD-ROM drive, tape drive, or removable storage disk. Communication device 560 can include any suitable device capable of transmitting and receiving signals over a network, such as a network interface chip or card. The components of the computer can be connected in any suitable manner, such as via a physical bus or wirelessly. Storage 540 can be a non-transitory computer-readable storage medium comprising one or more programs, which, when executed by one or more processors, such as processor 510, cause the one or more processors to execute methods described herein, such as all or part of any methods 200 described above with respect to FIG. 2.

Software 550, which can be stored in storage 540 and executed by processor 510, can include, for example, the programming that embodies the functionality of the present disclosure (e.g., as embodied in the systems, computers, servers, and/or devices as described above). In some embodiments, software 550 can be implemented and executed on a combination of servers such as application servers and database servers.

Software 550 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include, but is not limited to, an electronic, magnetic, optical, electromagnetic, or infrared wired or wireless propagation medium.

Computer 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer 500 can implement any operating system suitable for operating on the network. Software 550 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### EXAMPLE 1

In one example, 17,238 adverse event cases (Non-Serious Cases: 15034, Serious Cases: 2204) were analyzed. Each of the cases were manually classified and annotated by dedicated professional case managers. The cases were also reviewed by medical reviewers before being entered into a structured database for reporting to the FDA. The adverse event annotations were extracted into the Inside-Outside-Beginning (IOB) scheme which is commonly used in NER datasets such as CoNLL-2003. (See L. Ramshaw and M. Marcus, “Text Chunking Using Transformation-Based Learning,” In Proceedings of the Third ACL Workshop on Very Large Corpora, 1995, pages 8294.) The resulting annotation contained two entity labels: AE (adverse event) and AE LOC (anatomical position). The rest were labeled as O (outside, not belonging to any entities). The data was randomly divided with stratification to train (12,928 cases), validation (2,155 cases) and test subsets (2,155 cases). (The datasets that were analyzed are not publicly available due to privacy and security considerations.)

To experiment and evaluate the methodology, single tasks with dedicated network architectures were first tested on.

TABLE I
SERIOUSNESS CLASSIFICATION RESULTS WITH BASELINE
MODEL AND MULTI-TASK LEARNING MODEL

| Model | | Precision | Recall | F1-score | MCC |
|---|---|---|---|---|---|
| 2-Layer | Non-serious | 0.95 | 1 | 0.98 | 0.79 |
| Feedforward | Serious | 0.98 | 0.67 | 0.79 | |
| Network | Non-serious | 0.97 | 0.99 | 0.98 | 0.85 |
| MTL | Serious | 0.95 | 0.80 | 0.87 | |

TABLE II
NAMED ENTITY RECOGNITION RESULTS WITH BASELINE
MODEL AND MULTI-TASK LEARNING MODEL

| Model | Entity | Precision | Recall | F1-score |
|---|---|---|---|---|
| BiLSTM | O | 1.00 | 1.00 | 1.00 |
| | AE | 0.97 | 0.97 | 0.97 |
| | AE_LOC | 0.99 | 0.99 | 0.99 |
| MTL | O | 0.98 | 0.98 | 0.98 |
| | AE | 0.92 | 0.93 | 0.92 |
| | AE_LOC | 0.92 | 0.93 | 0.92 |

Then, an end-to-end multi-task learning network was trained, with two tasks in addition to the autoencoder: a) seriousness classification b) adverse event recognition (NER). Metrics were produced on the held-out test dataset. For inference and evaluation, the model will reproduce the input sequence, annotate the sequence with NER labels and classify the seriousness. For examples,

input: Patient developed blisters on the thigh region near the hip.

decode: Patient developed blisters on the thigh region near the hip.

ner_target: O O B-AE O O B-AE_LOC O O O B-AE_LOC O

ner_pred: O O B-AE O O B-AE_LOC O O O B-AE_LOC O

clf: 0

input: Patient had been hospitalized due to gastric bleeding and was just discharged.

decode: Patient had been hospitalized due to gastric bleeding and was just discharged.

ner_target: O O O B-AE O O B-AE_LOC B-AE O O O O

ner_pred: O O O B-AE O O B-AE_LOC B-AE O O O O O

clf: 1

Table I above shows that the multi-task learning model outperforms the baseline model, especially on serious cases recall. This performance improvement is also reflected in the Matthews correlation coefficient (MCC) score, which takes into account true and false positives and negatives.

Results from Table II show that BiLSTM is a very effective model, however Multi-Task Learning performance is also effective and not too far behind.

This Example demonstrates that MTL tasks prove to demonstrate robustness of the model architectures and techniques disclosed herein to handle at least the two tasks: NER and classification. This robustness may be due in part to the shared parameters within the encoder step, which is able to generalize across the two tasks. While the results for MTL task for classification is able to beat the baseline, the NER task for the baseline performed slightly better as shown in Table II. This may be due to the baseline LSTM model having to learn a smaller parameter space.

### EXAMPLE 2

The process of encoding adverse events to the MedDRA dictionary as a multi-class classification problem with ‘As Reported’ terms as the input and ‘Lower Level Terms (LLTs)’ as the target with approximately 21,000 distinct classes (LLTs) was modelled. Such high variation in the target variable led to very few training examples per class making it difficult for any machine learning model to learn from past data. To partially solve this problem, the ‘As Reported’, ‘To Be Coded’ and ‘Lower Level Terms’ were combined as the input variable and the corresponding LLTs as the target variable. This provided more training examples and helped capture more technical terms from the LLTs which otherwise couldn't be captured with just ‘As Reported’ terms.

Removal of non-alphanumeric characters: ‘As Reported’ terms often contained special characters like ‘−’, ‘/’, ‘!’, etc. whose usage patterns varied across case managers. Such variability in usage led to different vectored representations of similar ‘As Reported’ terms. Hence, dropping all the non-alphanumeric characters from ‘As Reported Terms’ helped to eliminate these differences.

Removal of stop words: Words like ‘a’, ‘an’, ‘at’, ‘and’, ‘be’, etc., usually referred to as ‘stop words’ are very common to majority of the ‘As Reported’ terms. These words add very little value in distinguishing one ‘As Reported’ term from other. Hence, these words were removed from the vocabulary entirely.

Lemmatization: Lemmatization is an algorithmic process of determining the lemma of a word based on its intended meaning. Lemmatization was performed to reduce morphological variations in the phrases to reduce the noise occurring because of usage of different forms of the same root word.

From the labeled dataset, the text-based data was converted into vectors in order to make the dataset machine readable. This enables selected features from the data to facilitate the machine learning process. The multiple text vectorization approaches were evaluated and TF-IDF vectorization approach was chosen as the right-fit for the K-Nearest Neighbor (KNN) model. The Deep Learning model is a more sophisticated approach that learns the features itself from the sequence of words (text input).

TF-IDF: Term Frequency-Inverse Document Frequency uses text vectorization technique to identify the importance of a word in a corpus. This is determined by the number of times the word appears in the document and is offset by the frequency of the documents containing the word in the corpus. TF-IDF automatically readjusts feature weights based on the number of times it appeared in the corpus.

K-Nearest Neighbor (KNN) Model: The K-nearest neighbor model is a non-parametric model used for classification and regression. It searches for k-closest examples in the training set which in turn are used for making the prediction. In case of a classification problem, it looks at the frequency of each of the target labels in these k examples and returns the class with highest frequency as the output.

Deep Learning: Deep Learning works off the premise that rather than spending time selecting significant features, a computer could use a very large dataset to instead identify patterns that exist in the dataset and apply a series of transformation to those input features until finally it outputs a single end classification score.

Multiple ML modeling approaches were evaluated, and two approaches were shortlisted: KNN model and Deep Learning to perform detailed experiments around feature engineering techniques and model architecture. Based on the performance, scalability, inference time and ease of deployment of the model, the Deep Learning model was chosen.

The steps involved in model training are:

1. Split the data into training and test sets (90-10 split)

2. Train the model

3. Evaluate training performance using performance metrics

Any machine learning model required the dataset to be divided into two sets:

Training set (90%)—to train the model

Test set (10%)—unseen data during ML training to determine the model performance

There was a significant class imbalance in the training and test sets. In order to reduce its impact on the model performance, the distribution of sub-classes within the training and test sets needs to remain the same. Hence, stratified sampling based on the Event Body System was performed.

For the Deep Learning model, the training data needed be split into a training and validation set. In each pass of the training data, the model is updated, and its performance is tested on the validation data. This process is repeated several times until no further improvement is obtained in the performance on the validation data.

The training data for Deep Learning model consisted of a sequence of tokens (words) from ‘As Reported’ terms as input (after removal of non-alphanumeric characters and converting all characters to lowercase) and the corresponding Lower Level Terms (LLTs) as target. A wide range of experiments were conducted with the model hyper parameters and model architectures to find the best setting for the model. The final model architecture is as described below and corresponds to the architecture shown in FIG. 4 herein.

The embeddings layer in the architecture internally learned the vectorized representations for characters. This was followed by two, one-dimensional (1D) convolutions (with different kernel sizes) which tend to capture sub-word information from the input text. Maxpooling is a sample-based discretization process. The objective is to down-sample an input representation (image, hidden-layer output matrix, etc.), reducing its dimensionality and allowing for assumptions to be made about features contained in the sub-regions binned. The maxpool operation highly activated the key sub-words suitable for making the prediction. The subsequent dense layers generated complex features by performing non-linear combinations over the maxpool output. Finally, the softmax layer generated a probability distribution over the LLTs. The LLTs corresponding to the top three scores were then able to be outputted, for example to be recommended to the user.

In order to have an efficient memory utilization and accelerate the process of training, the model was trained in batches of size 512 on GPU. Due to the very large number of trainable parameters in the network, it is often possible to overfit the model and hence regularization techniques are used. The techniques that were implemented in the above architecture are: L2 regularization and dropout. Regularization is a technique to discourage the complexity of the model. It does this by penalizing the loss function. This helps to solve the overfitting problem. Dropout refers to ignoring units (i.e., neurons) during the training phase of certain set of neurons which is chosen at random.

Performance of the Deep Learning based model was as follows:

TABLE III
AUTO-CODING DEEP LEARNING MODEL PERFORMANCE

| | Accuracy with | Accuracy with | Inference Time |
|---|---|---|---|
| | Exact Match | Ranking* | For 80k Records |
| Deep Learning | 66% | 0.82 | 2 min 55 sec |
| Model | | | |

“Accuracy with Ranking” was assessed based on whether the true LLT was in the list of top three LLTs predicted by the model; if so, that was considered as a correct prediction.

Model overfitting and underfitting was considered by the train-validation-test split. After the ML model was trained, the performance of the model was evaluated by analyzing the benchmarks with respect to the validation data. Model adjustments were made accordingly based off of these results, then a final benchmark was conducted using the testing dataset.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A pharmacovigilence adverse-event processing system comprising one or more processors configured to:
receive data comprising medical narrative text;
generate, based on the received data, using a recurrent neural network encoder, a fixed-length context vector representation of the medical narrative text;
query the fixed-length context vector representation, using a recurrent neural network decoder, to generate one or more hidden states;
process a first set of one or more of the one or more hidden states to generate an assessment of seriousness represented by the medical narrative text; and
process a second set of one or more of the one or more hidden states to generate a plurality of respective assessments of whether respective individual words of the medical narrative text correspond to one or more adverse events.

2. The system of claim 1, wherein generating the fixed-length context vector representation comprises using one or more distributed word representations.

3. The system of claim 1, wherein generating the fixed-length context vector representation comprises:
encoding the data comprising the medical narrative text using a word embedding to generate a vectorized representation of a plurality of individual words of the medical narrative text; and
encoding a sequence of the word embedding, using a recurrent neural network encoder, into the fixed-length context vector representation.

4. The system of claim 3, wherein the word embedding is pre-trained on a corpus of medical publication documents.

5. The system of claim 1, wherein the assessment of seriousness comprises a binary indication of whether a case is a serious case or a non-serious case.

6. The system of claim 1, wherein processing the first set of hidden states to generate the assessment of seriousness comprises:
processing the first set of hidden states through a plurality of fully connected layers to generate a fully connected layers output; and
processing the fully connected layers output using a first softmax function configured to generate a probability distribution indicating whether a case is serious or non-serious.

7. The system of claim 6, wherein the plurality of fully connected layers comprise at least one dense layer followed by at least one dropout layer.

8. The system of claim 1, wherein processing the first set of hidden states to generating the assessment of seriousness comprises applying a two layer feed-forward network with dropout.

9. The system of claim 1, wherein processing the second set of hidden states to generate the plurality of respective assessments of whether respective individual words of the medical narrative text correspond to one or more adverse events comprises performing a sequence labeling task using the second set of hidden states as an input sequence for the sequence labeling task.

10. The system of claim 9, wherein performing the sequence labeling task comprises determining, based on the input sequence, a label sequence having a highest probability.

11. The system of claim 9, wherein performing the sequence labeling task comprises using a decoded hidden state at a plurality of steps and the fixed-length context vector representation to determine a label for one of the plurality of individual words.

12. The system of claim 1, wherein processing the second set of hidden states to generate the plurality of respective assessments of whether respective individual words of the medical narrative text correspond to one or more adverse events comprises applying a second softmax function to the second set of hidden states of the recurrent neural network decoder.

13. The system of claim 1, wherein processing the second set of hidden states to generate the plurality of respective assessments of whether respective individual words of the medical narrative text correspond to one or more adverse events comprises applying a bidirectional Long short-term memory (LSTM) network.

14. The system of claim 1, wherein the one or more processors are configured to:
based on one or more of the individual words determined to correspond to one or more adverse events, generate a prediction of set of dictionary terms.

15. The system of claim 14, wherein generating the prediction of the set of dictionary terms comprises:
generating character embeddings based on the one or more of the individual words determined to correspond to one or more adverse events; and
process the character embeddings to generate the prediction of the set of dictionary terms.

16. The system of claim 15, wherein processing the character embeddings to generate the set of dictionary terms comprises:
processing the character embeddings through a set of parallel convolutional neural networks;
applying respective sample-based discretization processes to outputs of the parallel convolutional neural networks;
concatenating outputs of the discretization processes to generate concatenated data;
processing the concatenated data through a series of fully-connected layers; and
processing an output of the series of fully-connected layers through a third softmax function configured to generate the prediction of the set of dictionary terms.

17. The system of claim 1, wherein the one or more processors are configured to compute a total loss based on a sum of a first loss and a second loss, wherein the first loss quantifies loss associated with the assessment of seriousness the second loss quantifies loss associated with the plurality of respective assessments of whether respective individual words of the medical narrative text correspond to one or more adverse events.

18. The system of claim 17, wherein computing the total loss comprises computing the first loss using negative log-likelihood loss.

19. The system of claim 17, wherein computing the total loss comprises computing the second loss using masked cross entropy loss.

20. The system of claim 17, wherein:
the sum is further based on a third loss, wherein the third loss quantifies loss associated with the generation of the fixed-length context vector representation; and
computing the total loss comprises computing the third loss using masked cross entropy loss.

21. A pharmacovigilence adverse-event processing method performed at a system comprising one or more processors, the method comprising:
receiving data comprising medical narrative text;
generating, based on the received data, using a recurrent neural network encoder, a fixed-length context vector representation of the medical narrative text;
querying the fixed-length context vector representation, using a recurrent neural network decoder, to generate one or more hidden states;
processing a first set of one or more of the one or more hidden states to generate an assessment of seriousness represented by the medical narrative text; and
processing a second set of one or more of the one or more hidden states to generate a plurality of respective assessments of whether respective individual words of the medical narrative text correspond to one or more adverse events.

22. A non-transitory computer-readable storage medium storing instructions for pharmacovigilence adverse-event processing, the instructions configured to be executed by one or more processors of a system to cause the system to:
receive data comprising medical narrative text;
generate, based on the received data, using a recurrent neural network encoder, a fixed-length context vector representation of the medical narrative text;
query the fixed-length context vector representation, using a recurrent neural network decoder, to generate one or more hidden states;
process a first set of one or more of the one or more hidden states to generate an assessment of seriousness represented by the medical narrative text; and
process a second set of one or more of the one or more hidden states to generate a plurality of respective assessments of whether respective individual words of the medical narrative text correspond to one or more adverse events.
